SEQUENCE LISTING (8 sequences)

SEQ ID NO: 1 - protein, 178 aa, unidentified
MANKVLMAFVAADVLFAITGALILGYSLINQNTMNEVPTEGVQAAIRLSTKQFPLTAGVVNAVFIFITFLFTIPGMITPARGWLKVSGYMVTFCGVFSLIIGVYLWVLSLTTKADFAKLWISADPSVQELMQSAFQCCGYFNSTSPAFITDVQCPSPAAAALQRGCAAPVTSFVNVFL

SEQ ID NO: 2 - protein, 178 aa, unidentified
MVNKILATFVVADVLFLITGAVTLGFSLIARNNMFEQPDEGQQAARNLIYQKFPFEAGIANAAFIFITFVMTLPGIITPTRGWLKLAAYLITFCALFSLCVGVYLWVLTLTTQFDFFAIWIAQEPGVQNLMQTSFNCCGYFNSSSPAFVTNDLCSSPAAAALQRGCSGPVTSYSNILI

SEQ ID NO: 3 - protein, 179 aa, unidentified
MADKILLTYVILDILFVGSGALLLGFALTTKTGTSQAPTIASVATDLLLMGTPLNAAIGNAILIFFAFLISIPAMLLSTTRGWLKLHGFFVVVCGLFTLVIGLDIWFGTLESKQSLLDTWIAQSATTQSLLQEQLSCCGYFNSTSAPAFVIDSTCPNAIIAATMPGCSAAFVKLDGLFL

SEQ ID NO: 4 - protein, 179 aa, unidentified
MADKILWTYIILDLLFVGSGALLLGFALNTKAGTSQALSISSVGTKLLLEGTPLNAAIGNAILIFISFLLSIPAMTLSTTRGWLKLHGYMVVVCAIFTLVIGLDIWFGTLQSKESLLDTWNAQGPTTQSLLQEEFGCCGYFNSTSAPAFIIDSTCPNAIIAATMPGCSGAFVKFDGLFL

SEQ ID NO: 5 - protein, 178 aa, unidentified
MRDKLLLTYLVADVLFLGGGALILTVALTARDKIRSAPTLDNVAERLLLAHCPQLGEIINAGFVFFTFLLSIPAIIQSNDRIWMKIHGWMVVISGFITLIIGLIIWFLTLRTRSTLSDAWGNETPEVQSLLQQRFNCCGYLNSTSPPFQVDKTCPTDLDAAQKPGCVGPFSNFANSFL

SEQ ID NO: 6 - protein, 178 aa, unidentified
MPTKLMMVFVVMDLIFAGCGGLLLAFSLISEQTMRDSPTLDNVTQHILLGQCPLTAGVVNAIFVFVTFLLSLPALFLPTNRGWLRAQGWLVVFCATFTLGLGLTVWVETLQIRRNLSLLWGRETPLIQSLLQQKFDCCGYVNSTTPPFVQDSTCLNTLVAAQKGGCIAKFSSFANSYL

SEQ ID NO: 7 - protein, 178 aa, unidentified
MPTKLMMVFVGFDFLFAGCGGLLLGFSLMSEQSIRNTPSVDNVTQNLLLGQCPLTAGVVNAIFVFVTFLLSLPGLFLPTNRGWLRAQGWLVIICATFTLGLGLAIWLETLQTRKNLSNIWGRERPLIQSLLQQKFDCCGYTNSTTPPFVQDATCTNPLVAAQKGGCIGKFSSFANRFL

SEQ ID NO: 8 - DNA, 759 bp, unidentified
  source 1..759: mol_type = unassigned DNA
atggcggataaaattcttttgacatatgttattctggacatactctttgtaggttcaggtgcattattgcttgggtttgccttgacgacgaagactggtacttcgcaggctcctactattgccagtgtagctaccgatttgttgttgatgggtacacctttgaatggttggttttcccgtctattcattatattgttggtctgggaagaaagtatagcaatgagctaacatcactcgcaccctatagccgccattggaaatgcaatcttgatcttcttcgccttcctcatctccattcccgccatgctcctcagtaccactcgcggctggctcaaacttcatggcttcttcgtcgttgtctgtggtctcttcactctcgttatcggtcttgatatttggttcggtacactagaatctaaacaatctcttctcgatacatggattgcccaatctgcgaccacgcaaagtttgttgcaggaacaattaagctgttgtggatatttcaatagtacgagtgcgccagctttcgtaatcgatagtacttgtcctaatgctattattgcggccacaatgccgggatgttcagcggcatttgttaagttggatggattgttcttggatgtcatctttacagcggcttttggaatcgtgggcttggatgttgcgcttatttttggtattgcaatgcttaacaaggatcgtaaggagagggaaagatatagatttattgatgagaagaatggaacggggtctttttag

(19) United States
(12) Patent Application Publication
(10) Pub. No.: US 2024/0084302 A1
(43) Pub. Date: Mar. 14, 2024

(54) RNAi TARGETING OF FUNGAL PATHOGEN TETRASPANIN PROTEINS

(71) Applicant: The Regents of the University of California, Oakland, CA (US)

(72) Inventor: Hailing Jin, Riverside, CA (US)

(21) Appl. No.: 18/366,303

(22) Filed: Aug. 7, 2023

Related U.S. Application Data
(60) Provisional application No. 63/370,545, filed on Aug. 5, 2022.

(51) Int. Cl. C12N 15/113; C12N 15/82

(52) U.S. Cl. CPC C12N 15/113; C12N 15/8282; C12N 2310/14

(57) ABSTRACT

The disclosure provides RNAi strategies for targeting a fungal tetraspanin protein.

### CROSS-REFERENCE TO RELATED APPLICATIONS

This application claims benefit of priority to U.S. Provisional Application No. 63/370,545, filed Aug. 5, 2022, which is incorporated by reference in its entirety for all purposes.

### SEQUENCE LISTING

The instant application contains a Sequence Listing which has been submitted electronically in XML format and is hereby incorporated by reference in its entirety. Said XML copy, created on Nov. 6, 2023, is named 081906-1396498-250610US_SL.xml and is 9,900 bytes in size.

### STATEMENT AS TO RIGHTS TO INVENTIONS MADE UNDER FEDERALLY SPONSORED RESEARCH AND DEVELOPMENT

This invention was made with government support under grant no. R35GM136379 awarded by National Institute of General Medical Science. The government has certain rights in the invention.

### BACKGROUND

Fungal pathogens are a threat to global food security and can cause crop yield losses of up to 20% along with additional postharvest product losses of up to 10%. Currently, resistant strains of fungi to every major fungicide used in agriculture have been identified. In order to continue to safeguard global food security, novel strategies for combatting fungal pathogens must be developed. Recent advances have developed Spray-Induced Gene silencing (SIGS), where antifungal RNAs are applied to plant material through spray application. SIGS techniques utilize RNAi technology which allows the antifungal RNAs to be versatilely designed to be species specific and to target multiple genes simultaneously. SIGS has been successfully utilized to control a wide variety of fungal pathogens, insects, and viruses. A major bottleneck to SIGS approaches though is the identification of effective fungal gene targets for RNAi, which limits the variety and efficacy of dsRNA constructs that can be used.

### SUMMARY

Provided herein are nucleic acids, e.g., a dsRNA construct, that targets a virulence-related protein, PLS1, of fungal pathogens, e.g., Botrytis cinerea. PLS1 is a tetraspanin protein component of fungal extracellular vesicles. Although targeting PLS1 is described in technical section in the context of B. cinerea, analogous PLS1 proteins exist in other fungal pathogen species and can be similarly targeted.

In one embodiment, application of dsRNA, e.g., a dsRNA construct as described herein on plant material, such as leaves, can provide strong protection against B. cinerea. The compositions and method of the present disclosure can also be utilized in multiple ways where (1) plant material is treated directed with purified PLS1 dsRNA through spray or droplet application or (2) plant material is treated with bacteria expressing PLS1 dsRNA or (3) plant material is treated with vesicles loaded with PLS1 dsRNA.

### BRIEF DESCRIPTION OF DRAWINGS

FIG. 1: Sequence alignment of PLS1 proteins in various fungal species. Sequence alignment of B. cinerea PLS1 tetraspanin proteins against PLS1 proteins in other common fungal species including pathogens such as S. sclertiorum and V. dahliae. Cyan and capital letters indicate heavily conserved residues across species. Phylogeny tree indicating evolution of the PLS1 protein in fungal species. Species of PLS listed in upper panel (top to bottom): Colletotrichum lindemuthianum, Verticillium dahliae, Botrytis cinerea, Sclerotinia sclerotiorum, Coccidioides posadasil, Leptospheria maculans, Staganospora nodorum. Disclosed FIG. 1 sequences are SEQ ID NOS 1-7, respectively, in order of appearance.

FIG. 2A-B: BcPLS1 is important for B. cinerea virulence. (A). Arabidopsis thaliana leaves were inoculated with spores of wild-type B. cinerea (B05) or spores from the Δbcpls1 strain. Lesions were measured 2 days after inoculation. (B). Wild-type B. cinerea EVs partially complement the virulence of B. cinerea Δbcpls1 mutant. Δbcpls1 mutant spores were premixed with wild-type B. cinerea EVs before inoculation on Arabidopsis leaves. Lesions were measured 2 days after inoculation.

FIG. 3: Sequence of the B. cinerea PLS1 gene. The shaded region is the intron. The dsRNA construct targeting PLS1 in this illustrative example targets an exon region 3′ to the intron sequence (shown in red text). Disclosed FIG. 3 sequence is SEQ ID NO: 8.

FIG. 4A-B: Treatment of plant material with PLS1 dsRNA. (A) Droplet application of PLS1 dsRNA; (B) Spray application of PLS1 dsRNA.

### DESCRIPTION

### Introduction

RNA-based fungicides are an environmentally-friendly and easily customized alternative form of pesticides as compared to traditional chemicals. However, the number of effective dsRNA constructs is currently limited given the difficulty in identifying key pathogen genes for RNA interference. This invention provides a new target for RNAi efforts, specifically tetraspanin proteins, which are conserved across pathogenic fungal species.

The term “pathogen-resistant” or “pathogen resistance” refers to an increase in the ability of a plant to prevent or resist pathogen infection or pathogen-induced symptoms. Pathogen resistance can be increased resistance relative to a particular pathogen species or genus (e.g., Botrytis), increased resistance to multiple pathogens, or increased resistance to all pathogens (e.g., systemic acquired resistance). In some embodiments, resistance of a plant to a pathogen is “increased” when one or more symptoms of pathogen infection are reduced relative to a control (e.g., a plant in which a polynucleotide that inhibits expression of a fungal pathogen target gene is not expressed).

The term “nucleic acid” or “polynucleotide” refers to a single or double-stranded polymer of deoxyribonucleotide or ribonucleotide bases read from the 5′ to the 3′ end. Nucleic acids may also include modified nucleotides that permit correct read through by a polymerase and do not significantly alter expression of a polypeptide encoded by that nucleic acid.

The phrase “nucleic acid encoding” or “polynucleotide encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

In one aspect, the present disclosure provides a dsRNA construct that targets a tetraspanin protein, PLS1, of the fungal pathogen Botrytis cinerea. Many tetraspanin proteins, including TET8 in plant cells and CD63 in mammalian cells, are known to be specific markers for extracellular vesicles (EVs). In plant-pathogen interactions, these EVs have been shown to be important in facilitating cross-kingdom RNA interference. The present disclosure capitalizes on the role of the Botrytis tetraspanin proteins, such as PLS1, as a marker of B. cinerea EVs by using dsRNA that targets fungal tetraspanins (e.g. PLS1) to reduce B. cinerea virulence for spray induced gene silencing (SIGS) applications. In a further aspect, alternative tetraspanin proteins, e.g., Tetraspanin-like protein 1 in B. cinerea, can also be targeted.

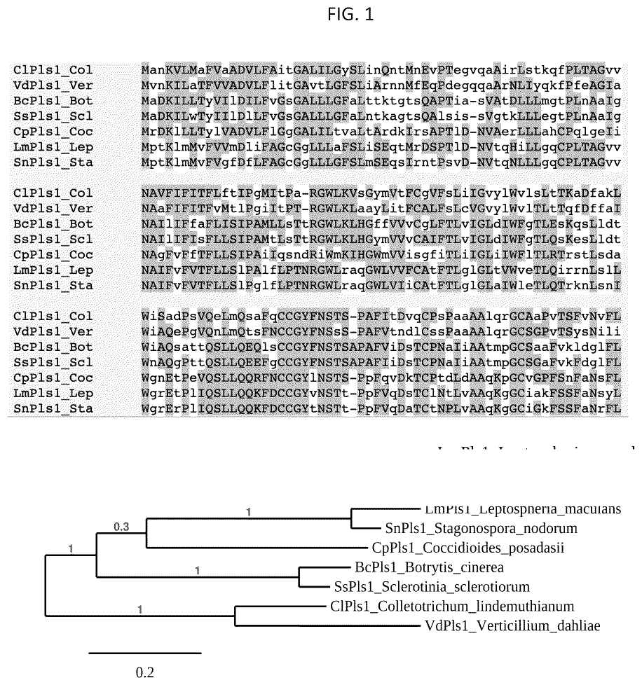

In a further aspect, tetraspanins such as PLS1 can be targeted using RNAi in other fungal pathogens. For example, PLS1 homologs have also been identified in other fungal pathogen species as shown in FIG. 1. Accordingly, the PLS gene of the fungal species listed in FIG. 1 can also be targeted using RNAi approaches, such as dsRNA.

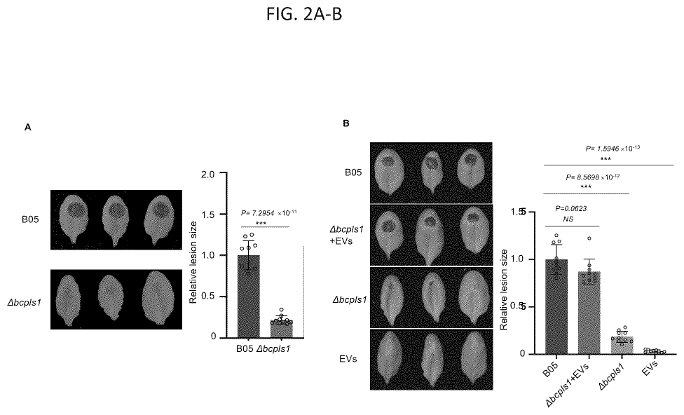

Supporting the importance of PLS1 polypeptide in fungal virulence, for B. cinerea virulence, a deletion mutant of PLS1 in B. cinerea (Δbcpls1) exhibits reduced virulence as compared to wild-type B. cinerea (B05) (FIG. 2A) and addition of EVs derived from wild-type B. cinerea to Δbcpls1 spores recapitulates the virulence phenotype (FIG. 2B).

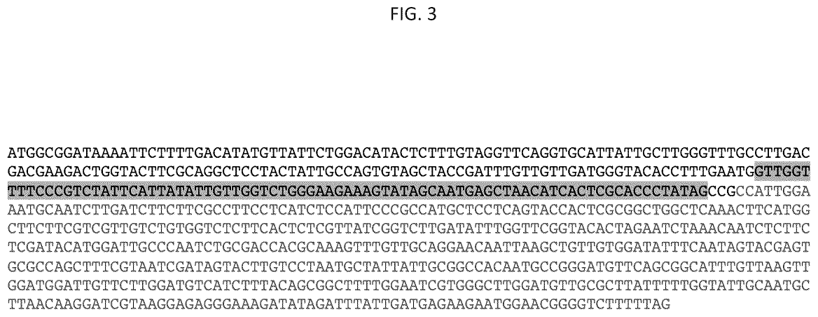

An illustrative dsRNA construct is an RNA construct that contains a sequence that is complementary to 500 bp of the B. cinerea PLS1 sequence shown in FIG. 3. As appreciated by one of skill in the art, however, alternative dsRNA sequences that are longer or shorter than the 500 bp can be used, and/or combinations of different regions within the PLS1 gene can be targeted.

dsRNA can be prepared by in vitro transcription. However, alternative methods can be employed, e.g., using bacterial or viral production.

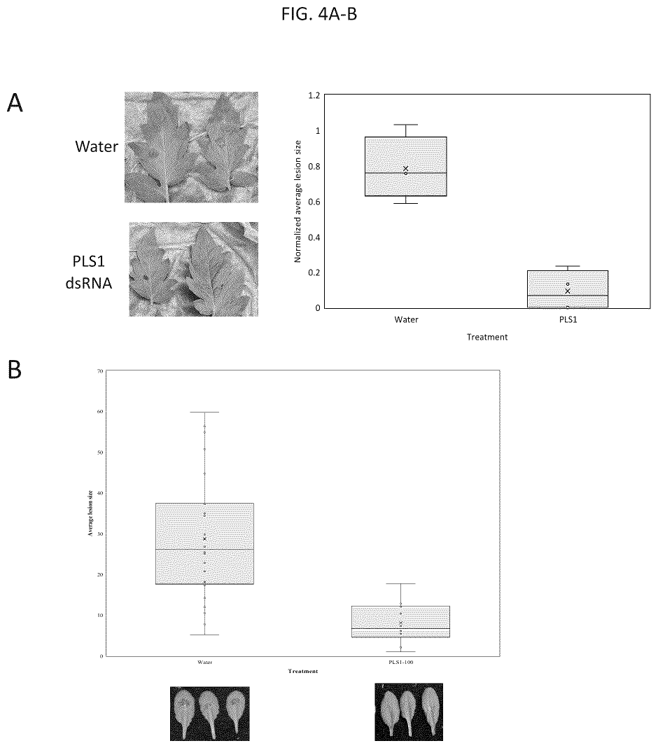

In one embodiments, droplet application of the PLS1 dsRNA construct on plant material, such as leaves, provided strong protection against B. cinerea as compared to water (see, e.g., FIG. 4A).

Spray application of the PLS1 dsRNA onto Arabidopsis leaves also provided significant protection against B. cinerea infection (FIG. 4B).

In some embodiments, plant material is treated directly with the purified PLS1 dsRNA construct through spray or droplet application. In other embodiments, plant material is treated with bacteria expressing the PLS1 dsRNA. In still other embodiments, plant material is treated with vesicles loaded with the PLS1 dsRNA construct.

In a further aspect, tetraspanin proteins such as PLS1 have been identified as EV components in a wide range of fungal species. Accordingly, in some embodiments, tetraspanin proteins such as PLS1 can targeted with RNAi strategies, e.g., dsRNA applied to a plant, for other fungal pathogens such as Sclerotinia sclerotiorum.

In some embodiments a plant may be genetically modified to express inhibitory RNA molecules that target a fungal pathogen PLS1.

In some embodiments, an inhibitory RNA employed to target a fungal tetraspanin gene such as PLS1, targets a sequence of at least 15, 20, 30, 40, 50, 60, 70, 80, 90, 100, 150, 200, 250, 300, 350, 400, 450, 500 or more contiguous nucleotides of the sequence shown in FIG. 3, or the sequence of a homologus fungal PLS1 gene). In some embodiments, the polynucleotide comprises a nucleic acid having a sequence that is identical or complementary to at least 15, 20, 25, 30, 35, 40 or more contiguous nucleotides of PLS1 sequence shown in FIG. 3 or a sequence of a homologus fungal PLS1 gene. In some embodiments, the polynucleotide comprises a double-stranded nucleic acid having a sequence that is identical or substantially identical (at least 60%, 65%, 70%, 75%, 80%, 85%, 90%, 91%, 92%, 93%, 94%, 95%, 96%, 97%, 98%, or 99% identical) to the PLS1 sequence shown in FIG. 3, or a homologous fungal PLS1 gene, or a fragment thereof (e.g., at least 15, 16, 17, 18, 19, 20, 21, 22, 23, 24, 25, 26, 27, 28, 29, 30, 31, 32, 33, 34, 35, 36, 37, 38, 39, 40, at least 50, at least 60, at least 70, at least 80, at least 90, at least 100, at least 150, at least 200, at least 250, at least 300, at least 350, at least 400, at least 450, or at least 500 contiguous nucleotides thereof). In some embodiments, the polynucleotide comprises an inverted repeat of a fragment (e.g., at least 15, 20, 25, 30, 35, 40 or more contiguous nucleotides) of a PLS1 sequence shown in FIG. 3 or a homologous fungal PLS1 gene, and further comprises a spacer region separating the inverted repeat nucleotide sequences.

One or more features from any embodiments described herein or in the figures can be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications can be made thereto without departing from the spirit or scope of the appended claims.

What is claimed:

1. A method of increasing fungal pathogen resistance in a plant or a part of a plant, the method comprising:
applying a dsRNA or RNA (sRNA) duplexes that target a fungal tetraspanin gene to a plant or part of a plant, wherein the plant or the part of the plant has increased resistance to a fungal pathogen compared to a control plant or control plant part that has not been contacted with the dsRNAs or sRNA duplexes.

2. The method of claim 1, wherein the fungal pathogen gene is a PLS1 gene.

3. The method of claim 1 or 2, wherein the pathogen is Botrytis, Verticillium, or Sclerotinia.

4. The method of claim 1, 2, or 3, wherein the dsRNA or sRNA are contained within liposomes or EVs.

5. The method of claim 1, 2, or 3, wherein the dsRNA or sRNA is applied by contacting the plant or part of the plant with bacteria that express the dsRNA or sRNA.

6. The method of claim 1, 2, or 3, wherein the dsRNA or sRNA is sprayed onto the plant.
